(19) United States
(12) Patent Application Publication
(10) Pub. No.: US 2012/0310211 A1
(43) Pub. Date: Dec. 6, 2012

(54) PHENTERMINE LIQUID DOSAGE FORM

(75) Inventors: Leneri Du Toit, Pretoria (ZA); Eduan Stoltz, Pretoria (ZA); Marina Coetzee, Pretoria (ZA)

(73) Assignee: Pharmacorp CC, Pretoria (ZA)

(21) Appl. No.: 13/315,579

(22) Filed: Dec. 9, 2011

Related U.S. Application Data
(63) Continuation-in-part of application No. PCT/ZA2010/000026, filed on May 31, 2010.

(30) Foreign Application Priority Data
Jun. 12, 2009 (ZA) 2009/4136

(51) Int. Cl. A61K 31/137; A61J 7/00; A61P 3/04

(52) U.S. Cl. 604/514; 514/654; 604/246

(57) ABSTRACT

A pharmaceutical composition liquid dosage form of phentermine is provided, the dosage form including a liquid carrier and suspended or dissolved therein one or more pharmaceutically active ingredient selected from phentermine, salts and bases thereof. Also provided is a method of administering phentermine to an obese patient in need thereof and for appetite suppression or related conditions.

### CROSS-REFERENCE TO RELATED APPLICATIONS

This application is a continuation-in-part, under 35 U.S.C. §120, of International Patent Application No. PCT/ZA2010/000026, filed on May 31, 2010 under the Patent Cooperation Treaty (PCT), which was published by the International Bureau in English on Mar. 24, 2011, which designates the United States and claims the benefit of South Africa Application No. 2009/4136, filed Jun. 12, 2009, the disclosures of which are hereby expressly incorporated by reference in their entireties and are hereby expressly made a portion of this application.

### FIELD OF THE INVENTION

Pharmaceutical compositions for the oral liquid dosage form and administration of phentermine, an α,α-dimethyl phenethylamine (phenyl-tertiary-butylamine) are provided.

### BACKGROUND TO THE INVENTION

To date, phentermine has only been available in solid dose oral dosage format in capsules and tablets. These capsules and tablets contain phentermine in a base or HCl salt or as a resin compound in dosages ranging from 8-40 mg.

### SUMMARY OF THE INVENTION

There is a need for a dosage form of phentermine which can be more accurately dosed so that it is possible to deliver a dosage accurately tailored to the needs of a patient to allow for maximum efficacy of appetite suppression while minimizing side effects and minimizing the need to have various dosage form strengths (15 mg and 30 mg and, for example, 40 mg packs of capsules containing phentermine or a salt or resin of phentermine) with one single format product with the ability to deliver these dosages and various others without the multiple pack costs and associated regulatory complexities.

According to a first aspect, there is provided a pharmaceutical composition liquid dosage form of phentermine, said dosage form including a liquid carrier and suspended or dissolved therein one or more pharmaceutically active ingredient selected from phentermine, salts and bases thereof.

The composition may include a pro drug of phentermine.

The liquid carrier may be water, oil, or alcohol or a combination of thereof so that the dosage form is a liquid dosage form.

The liquid carrier may be an oil.

The liquid carrier may include one or more alcohol, for example, ethanol.

The liquid dosage form may include elixirs, liquids, solutions, suspension in an aqueous liquid or a non-aqueous liquid, emulsions, liquid dispersions for oral administration including syrups, emulsions, solutions, and suspensions.

The liquid dosage form may include one or more of the HCl salt of phentermine, otherwise known as phentermine HCl, and any other salts of phentermine.

The phentermine may be covalently bound to a chemical moiety, wherein said chemical moiety provides release of phentermine at a rate where the level of phentermine is within a therapeutic range but below toxic levels over an extended periods of time, e.g., 8-24 hours or greater.

The concentration of phentermine in the liquid carrier may be selected such that the amount of milligrams of phentermine, its salt, or base, which is to be administered, can easily and accurately be determined from the number of milliliters or measured drops of the pharmaceutical composition administered.

The concentration may be from 10 to 100 mg phentermine per 1 ml of liquid dosage form. Typically, the concentration may be from 15 mg/ml to 40 mg/ml, for example, 30 mg/ml.

The liquid dosage form may include surface-active agents selected from a draught, in water or a syrup, in a non-aqueous suspension wherein suspending agents may be included, or in a suspension in water or a syrup.

The liquid dosage form may, where desirable or necessary, include flavouring, preserving, suspending, thickening or emulsifying agents.

According to a second aspect, there is provided a phentermine containing liquid composition administration apparatus, which apparatus includes a container and a dropper sized and dimensioned to permit the phentermine containing liquid dosage form of the various embodiments to be accurately administered dropwise in from 1 to 50 mg phentermine per administration thereof.

The unexpected and new method of delivering a required phentermine or phentermine HCl dose is by the counting of the number of drops that will deliver a specific concentration of phentermine or phentermine HCl per drop. The Medical practitioner may therefore instruct the patient to take for example ten (10) drops of the liquid containing 1.5 mg per drop if he requires the patient to be administered 15 mg as explained below.

The dropper may thus be calibrated so that 20 drops of the phentermine containing liquid dosage form are 1 ml, which is equivalent to from 1 to 100 mg phentermine per ml.

Thus, in order to administer 15 mg of phentermine only 10 drops are required of a 30 mg/ml phentermine concentration liquid dosage form and to administer 30 mg of phentermine 20 drops are required.

The container may be from 10 ml to 100 ml in size, typically 30 ml.

The container may be an amber Type III Ph Eur glass bottle with a polypropylene screw-on cap with a dropper insert that will allow for accurate droplet administration.

The container may also be of any other material that will allow the phentermine liquid dosage form to be pharmaceutically stable and accurately dosed.

Besides glass, the container may be made entirely or in part of polyethylene, HDPE, polypropylene or any other suitable material that will allow accurate dosing and stability and is inert enough not to leach chemicals into the liquid dosage form.

According to a third aspect, there is provided a method of administering phentermine, or salts or bases thereof, to a patient in need thereof, said method including administering a required number of drops of a liquid dosage form of the various embodiments of phentermine, phentermine salt, or phentermine base containing composition to the patient, thereby administering the required dose to the patient.

The method may comprise administering 10 drops, equivalent to 0.5 ml, of a 30 mg/ml composition once or twice daily, or variations of the dosage according to the patient's individual requirements or any number of drops to deliver the required daily therapeutic dose suitable to the patient.

The method may include administering the liquid dosage form of the various embodiments to a patient to be treated for obesity or in need of appetite suppression, or related conditions.

The method extends to a method of treatment of obesity, appetite suppression, or related conditions by administering therapeutically effective amounts of phentermine periodically to a patient by means of a liquid dosage form thereof.

The method of treatment extends to the dropwise administration of the phentermine liquid dosage form at concentrations that make specific accurate dosing thereof possible.

phentermine is an anorectic. Anorectics are used to decrease appetite by possibly changing brain levels of serotonin and dopamine and may also stimulate hypothalamic neurons to release norepinephrine that may cause appetite suppression by increasing blood leptin levels and a decrease in neuropeptide Y production, which may result in increased satiety and decrease appetite. Phentermine is a nervous system stimulator causing stimulation, elevation of blood pressure, and faster heart rates. Phentermine in its current dosage forms is often poorly tolerated.

Obesity, typically defined as 20% over ideal weight results, is viewed as a contributor factor to an increase in certain diseases including high cholesterol levels, heart disease, high blood pressure, gallbladder disease, type II diabetes mellitus, hardening of the arteries, and degenerative arthritis. Controlling and decreasing an individual's weight typically results in decreases in blood pressure, cholesterol levels, and an improvement in diabetes control. A well-tolerated, safe and effective weight loss therapy that can minimise obesity-associated co morbidities is needed to impede the growing global obesity epidemic.

Phentermine is currently available on a prescription by a qualified and authorised medical practitioner in both name brand and generic versions. Market doses include 8 mg tablets, 15 mg capsules, 30 mg capsules and 40 mg phentermine resin and 15 mg and 37.5 mg tablets and capsules of phentermine HCl salts. Phentermine is generally stored in a tight container at room temperature. Phentermine is typically prescribed as a short-term drug accompanied by a diet and behaviour modification/exercise routine to treat obesity. Although, some programs combine it with diet and life style modifications and exercise over longer terms in selected obese patients.

Phentermine also has several potential side effects including diarrhea, dry mouth, constipation, an unpleasant taste, hives, impotence, palpitations, high blood pressure, fast heart rates, overstimulation, insomnia, restlessness, tremor, and dizziness. In addition, phentermine is potentially addicting. It is important that the dosage of phentermine be controlled to minimise adverse side effects.

One way in which to regulate the concentration of phentermine in the body is by attaching it to an ion exchange resin format to control the release in the body that then regulate the release of phentermine from the resin. These dosage forms are in 15, 30 or even 40 mg dosage form. The other immediate dosage forms are available in phentermine HCl ranging from 10 mg to 37.5 mg phentermine containing tablets or capsules.

All these dosage forms are not able to tailor the amount of phentermine in a usage and release rate that will be suitable and individualised for each patient to the accuracy of 1.5 mg variance. The various embodiments allow for the individualised dosage regime per patient to achieve optimum appetite suppression and minimal side effect ratio.

The effective delivery of phentermine or its salt is often critically dependent on the delivery system used. Phentermine (non salt but base) C₁₀H₁₅N=149.2 is a colorless oily liquid that is slightly soluble in water: soluble in alcohol, chloroform and ether, while phentermine HCl C₁₀H₁₅N.HCl=185.7 is a white odorless crystalline powder that is very soluble in water, alcohol and chloroform and insoluble in ether. Surprisingly, it has been discovered that a formulation can be prepared where the solubility of phentermine in a salt form (HCl) could be achieved and be stable over a shelf life period. The importance of these systems becomes magnified when patient compliance and of phentermine stability are taken under consideration.

The formulation of phentermine in a liquid drop markedly improves the safety of that drug, dose adjustments and minimising side effects. In general, increasing the stability of phentermine, such as prolonging shelf life will assure dosage reproducibility.

The various embodiments provide drug formulations that effectively deliver phentermine in tailored and adjustable patient specific dosage form more accurate than the current tablets or capsules formats containing 15, 30, 37.5 or 40 mg phentermine or its salts. The various embodiments provide methods of protecting and controlling the delivery and/or release of phentermine.

The various embodiments enable the use of new phentermine compositions that can reduce the technical, regulatory, and financial risks associated with phentermine agents while improving their reproducibility, bioavailability, reliability, dosage and patient customisation.

The compounds of the various embodiments may be provided in several useful liquid forms. As such, improved methods are provided to make pharmaceutically effective phentermine compounds, compositions and methods of using the same with reduced potential for overdose and/or reduced side effects in an oral drop form with a calibration mechanism.

The various embodiments relate to the pharmaceutical presentation of phentermine through dissolving, suspending, binding, emulsifying, or in some other manner providing it in liquid dosage form and presenting it in a drop dosage delivery system that will impact the accuracy, tolerability and flexibility of the dosage given, the rate of absorption, the extent of absorption, the metabolism, the distribution, and the elimination (ADME pharmacokinetic properties) of phentermine. As such, the alteration of one or more of these characteristics may be designed to provide fast or slow absorption and release. Additionally, alteration of one or more of these characteristics may reduce the side effects associated with taking phentermine.

The delivery of phentermine in this tailored liquid dosage form provides bioavailability but reduces the occurrence and severity of side-effects and possible overdosage from high concentrations observed at Cmax.

The liquid dosage form of various embodiments provides for reduction in the potential for overdosing as well as improvement in phentermine's characteristics with regard to high toxicities or suboptimal release and absorption profiles.

The various embodiments provide a method for delivering phentermine to a patient, the patient being a human or a non-human animal, comprising administering to the patient pharmaceutically effective doses of phentermine compositions in the liquid dosage form of the various embodiments.

It is believed that the liquid dosage forms, methods, apparatus, compounds, and compositions of the various embodiments provide important advantages and advances. The methods and compositions of the various embodiments are believed to prevent and/or avoid overdosing (e.g., “spiking”). By assuring dosage reproducibility and/or reducing dosage availability, the various embodiments provide the added advantage of improving patient compliance and minimising side effects and adjusting the dosage in single drop steps of up to 1 drop equals 1.5 mg accuracy. The various embodiments are also believed to provide time-release properties to phentermine. Providing time-release properties also assures dosage reproducibility.

In a currently preferred embodiment, the time-release properties provided by the various embodiments are not dependent upon other commonly used delay release or time-release formulations, such as a microencapsulating matrix or resin acidification during manufacturing. This provides a further advantage of reliable dosing and batch-to-batch reproducibility. This embodiment provides a further advantage of time-release properties without heightened dependence on water solubility of the phentermine. As such, the time-release properties do not require further formulations such as the dissolution process involved in an enteric coated active agent controlled by pH. Dosing and the desired blood levels or therapeutic levels can be achieved by the ability to deliver different doses in time related intervals because of the flexibility and accurate dosages able to be given by the drop dosage system not previously available for phentermine or its salts.

Another advantage provided by various embodiments is the control of phentermine dose delivery system with regard to mg per dose and time of delivery or combinations thereof. The control of these physical characteristics enables predictable diffusion rates and pharmacokinetics and minimises side effects and specific dosage per individual patient needs. This liquid dosage form in a drop format may be administered to a patient to treat obesity or to suppress appetite, or related conditions.

The various embodiments provide the amount of biologically available phentermine in a regulated manner and therefore, side effects known from taking too high a dose of phentermine can be prevented. The amount of free phentermine is regulated by the mechanism that allows for accurate patient adjusted dose of phentermine thereby minimizing the potential for adverse side effects from high doses. In addition, the absorption of phentermine may be improved since phentermine or phentermine HCl is administered in a liquid form.

The various embodiments provide several benefits for phentermine administration, such as but not limited to longer shelf life, prolonged pharmacologic effect through delayed release of phentermine; phentermine can be combined together or with adjuvants to produce synergistic effects; enhanced absorption of the phentermine in the intestinal tract; and formulation for digestion by intestinal enzymes, intracellular enzymes or blood serum enzymes.

Patients that can not swallow capsules and tablets can be treated with the liquid dosage form. The liquid can be added to other substances and in combination with other substances that can play a role in the treatment of obesity if needed.

Compositions of the various embodiments may comprise the formation of different carrier systems ranging from water to oil to alcohol. Throughout the application it is intended to describe the general dissolving, suspending, emulsifying, or in any other manner obtaining and stabilising phentermine in a liquid dosage format.

These products will be used at levels similar to those used in treating obesity patients with current treatments. Determining the precise levels to be used in a particular patient may be accomplished using methods well known to those of skill in the art. The compositions will be particularly useful in providing oral liquid dosage formulations.

Another embodiment is a method for safely delivering phentermine comprising providing a therapeutically effective amount of phentermine in liquid dosage form, whether in solution or suspension. Another embodiment may also provide a means for reducing drug toxicity by altering the rate of clearance of phentermine.

Another embodiment is a composition or method for a sustained-release phentermine composition comprising providing phentermine which has been covalently bound to a chemical moiety, wherein said chemical moiety provides release of phentermine at a rate where the level of phentermine is within the therapeutic range but below toxic levels over an extended periods of time, e.g., 8-24 hours or greater.

Another embodiment is a composition or method for preventing a Cₘₐₓ spike and/or providing a more consistent release curve for phentermine while still providing a therapeutically effective bioavailability curve comprising phentermine that has been dissolved in the carrier and delivered in the specific dosage at the desired rate.

In accordance with the various embodiments and as used herein, the following terms are defined with the following meanings, unless explicitly stated otherwise.

The dosage forms, compounds, compositions and methods of the various embodiments utilize “phentermine,” which are also referred to as phentermine salts, phentermine bases, or pharmaceutically active ingredients.

Throughout this application the use of “chemical moiety” is meant to include any chemical substance, naturally occurring or synthetic that dissolves or binds phentermine until the phentermine is released and absorbed.

Throughout this application the use of “carrier” is meant to include the liquid in which phentermine is dissolved or suspended so that it can be accurately delivered in the liquid dosage form by a dropper.

Cₘₐₓ is defined as the maximum concentration of free phentermine in the body obtained during the dosing interval.

Tₘₐₓ is defined as the time to maximum concentration.

Cₘᵢₙ is defined as the minimum concentration of phentermine in the body after dosing.

t_{1/2} is defined as the time required for the amount of phentermine in the body to be reduced to one half of its value.

“Patient” as used herein, refers broadly to any human or animal that is in need of treatment, most preferably and animal or human that is obese. The patient may be a clinical patient such as a human or a veterinary patient such as a companion, domesticated, livestock, exotic, or zoo animal. Animals may be mammals, reptiles, birds, amphibians, or invertebrates.

“Mammal” as used herein, refers broadly to any and all warm-blooded vertebrate animals of the class Mammalia, including humans, non-human primates, felines, canines, pigs, horses, sheep, etc.

“Treating” or “treatment” as used herein, refers broadly to preventing the disease, i.e., causing the clinical symptoms of the disease not to develop in a patient that may be exposed to or predisposed to the disease but does not yet experience or display symptoms of the disease, inhibiting the disease, i.e., arresting or reducing the development of the disease or its clinical symptoms, and/or relieving the disease, i.e., causing regression of the disease or its clinical symptoms. Treatment also encompasses an alleviation of signs and/or symptoms.

“Therapeutically effective amount” as used herein, refers broadly to the amount of a compound that, when administered to a patient for treating obesity is sufficient to effect such treatment for obesity or appetite suppression, or related conditions. The “therapeutically effective amount” will vary depending on the compound, the disease and its severity and the age, weight, etc., of the patient to be treated. “Effective dosage” or “effective amount” of the phentermine compound or composition is that which is necessary to treat or provide prophylaxis for obesity or appetite suppression, or related conditions.

“Diagnosis” as used herein, refers broadly to the practice of testing, assessing, assaying, and determining whether or not a patient is obese. In particular, one criterion may be the percentage of body weight due to fat.

For each of the embodiments recited herein, the carrier may comprise of one or more of the following sterile water, sterile oil, or alcohol or combination of other liquids in which phentermine dissolves or may be suspended.

The phentermine active ingredient may also be in salt form. Pharmaceutically acceptable salts, e.g., non-toxic, inorganic and organic acid addition salts, are known in the art. Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, isothionate, lactate, lactobionate, laurate, laurylsulphonate, malate, maleate, mandelate, mesylate, methanesulfonate, methylsulfate, mucate, naphthylate, napsylate, nicotinate, nitrate, N-methylglucamine ammonium salt, oleate, oxalate, palmitate, pamoate, pantothenate, pectinate, phosphate, phosphateldiphosphate, picrate, pivalate, polygalacturonate, propionate, p-toluenesulfonate, saccharate, salicylate, stearate, subacetate, succinate, sulfate, sulfosaliculate, strontium, suramate, tannate, tartrate, teoclate, thiocyanate, tosylate, triethiodide, undecanoate, and valerate salts, and the like.

In the various embodiments, phentermine may be dissolved or suspended in any liquid safe to the human body that will accurately deliver the dosage in the desired effective therapeutic amount accurate up to 1 drop equals 1.5 mg. Various drop sizes are contemplated, as are various amounts of phentermine per drop.

In addition to the phentermine the various embodiments may further comprise one or more pharmaceutical additives. Pharmaceutical additives include a wide range of materials including, but not limited to diluents and bulking substances, binders and adhesives, lubricants, glidants, plasticizers, disintegrants, carrier solvents, buffers, colorants, flavourings, sweeteners, preservatives and stabilizers, adsorbents, and other pharmaceutical additives known in the art.

Lubricants include, but are not limited to, magnesium stearate, calcium stearate, zinc stearate, powdered stearic acid, glyceryl monostearate, glyceryl palmitostearate, glyceryl behenate, silica, magnesium silicate, colloidal silicon dioxide, titanium dioxide, sodium benzoate, sodium lauryl sulfate, sodium stearyl fumarate, hydrogenated vegetable oil, talc, polyethylene glycol, and mineral oil.

Surface agents for formulation include, but are not limited to, sodium lauryl sulfate, dioctyl sodium sulfosuccinate, triethanolamine, polyoxyethylene sorbitan, poloxalkol, and quarternary ammonium salts; excipients such as lactose, mannitol, glucose, fructose, xylose, galactose, sucrose, maltose, xylitol, sorbitol, chloride, sulfate and phosphate salts of potassium, sodium, and magnesium; gelling agents such as colloidal clays; thickening agents such as gum tragacanth or sodium alginate, effervescing mixtures; and wetting agents such as lecithin, polysorbates or laurylsulphates.

Colorants can be used to improve appearance or to help identify the pharmaceutical composition. Exemplary colorants include D&C Red No. 28, D&C Yellow No. 10, FD&C Blue No. 1, FD&C Red No. 40, FD&C Green #3, FD&C Yellow No. 6, and edible inks.

Flavourings improve palatability and may be particularly useful for liquid dosage forms. Flavourings include, but are not limited to maltol, vanillin, ethyl vanillin, menthol, citric acid, fumaric acid, ethyl maltol, and tartaric acid. Sweeteners include, but are not limited to, sorbitol, saccharin, sodium saccharin, sucrose, aspartame, fructose, mannitol, and invert sugar.

Preservatives and/or stabilizers improving storage stability include, but are not limited to, alcohol, sodium benzoate, butylated hydroxy toluene, butylated hydroxyanisole, and ethylenediamine tetraacetic acid.

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc.

In embodiments where the pharmaceutical composition is formulated for the liquid dosage form, the pharmaceutical composition may include one or more solvents, emulsion or suspension liquids. Suitable liquids and solvents include, but are not limited to, water; alcohols such as ethanol and isopropyl alcohol; methylene chloride; vegetable oil; polyethylene glycol; propylene glycol; and glycerine, and mixtures and combinations thereof.

The pharmaceutical composition can comprise a buffer. Buffers include, but are not limited to, lactic acid, citric acid, acetic acid, sodium lactate, sodium citrate, and sodium acetate.

However, it should be noted that the phentermine in this dosage form controls the release of phentermine into the digestive tract over a period of time resulting in an improved profile when compared to immediate release and sustained release tablet or capsules and reduces and/or prevents toxicity without the addition of usual additives. In a preferred embodiment no further sustained release additives are required to achieve a blunted or reduced pharmacokinetic curve while achieving therapeutically effective amounts of phentermine release.

The dose range for adult human beings will depend on a number of factors including the age, weight and condition of the patient and the administration route. Tablets and capsules and other forms of presentation provided in discrete units contain a daily dose, or an appropriate fraction thereof, of the phentermine. This liquid dosage form can contain a dose of about 1.5 mg per drop to a maximum dose of equivalent to the highest regulatory approved dosage of phentermine. The amount of phentermine per drop can range from 0.1 or less to 10 or more mg per drop, e.g., from 0.5 to 2 mg per drop.

The drop liquid dosage forms provided in discrete units can contain a daily dose, or an appropriate fraction thereof, of phentermine or phentermine salt or a pro-drug.

The amount of liquid in a drop can be selected as desired. There are various definitions of a “drop” in terms of the volume of liquid contained therein, each of which is encompassed within the preferred embodiments. For example, the definitions include: the metric drop, 1/20 mL (50 μL); the medical drop, 1/12 mL (83⅓% μL); the Imperial drop, 1/36 of a fluidram ( 1/288 of an Imperial fluid ounce, or 1/1440 of a gill) (approximately 99 μL); 1/1824 of a gill (approximately 78 μL); the U.S. drop, 1/60 of a US fluidram, 1/80 of a teaspoon or 1/480 of a U.S. fluid ounce (approximately 62 μL); or 1/96 of a teaspoon or 1/576 US fl oz (approximately 51 μL). Typical drops per unit volume can range from 5 or less drops/mL to 60 or more drops/mL, e.g., 20 drops/mL.

The formulations may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24-hour period. The doses may be uneven doses with regard to one another or with regard to the individual components at different administration times. Preferably, a single dose is administered once daily in the morning adjusted to maximise effect and minimise side effects. Dosing may also be adjusted to alternative days and an intermediate treatment regime specific to an individual patient.

The liquid dosage form of the various embodiments may be provided in different glass size bottles and a calibrated drop dispenser. Further, the compositions of the various embodiments may further include or be accompanied by indicia allowing individuals to identify the compositions as products for a prescribed treatment. The indicia may further additionally include an indication of the above specified time periods for administering the compositions. For example the indicia may be time indicia indicating a specific or general time of day for administration of the composition, or the indicia may be a day indicia indicating a day of the week for administration of the composition.

The liquid compounds of the various embodiments can be administered by a variety of liquid dosage forms. Any biologically acceptable dosage form known to persons of ordinary skill in the art, and combinations thereof, are contemplated. Examples of such liquid dosage forms include, without limitation elixirs, liquids, solutions, suspension in an aqueous liquid or a non-aqueous liquid, emulsions, liquid dispersions for oral administration (e.g., syrups, emulsions, solutions or suspensions).

However, the most effective means for delivering the phentermine compounds of the various embodiments is orally, to permit maximum release of phentermine to provide therapeutic effectiveness. When delivered by the oral route phentermine is released into circulation, preferably over in more specific dosage regime due to the drop applicator.

The smaller size of the phentermine dose per drop promotes ease of swallowing.

For oral administration surface-active agents may be presented in a draught, in water or a syrup, in a non-aqueous suspension wherein suspending agents may be included, or in a suspension in water or a syrup. Where desirable or necessary, flavouring, preserving, suspending, thickening or emulsifying agents can be included.

It will be appreciated that the pharmacological activity of the compositions of the various embodiments can be demonstrated using standard pharmacological models that are known in the art. For each of the described embodiments one or more characteristics as described throughout the specification may be realized. It should also be recognized that the compounds and compositions described throughout the specification may be utilized for a variety of novel methods of treatment, reduction of toxicity, improved release profiles, etc.

It is an advantage of the various embodiments that it makes the number of independently manufactured, stored, prescribed, and dispensed oral dosage forms redundant since with this embodiment various dosages can be tailored to every patient by varying the number of drops thereby reducing the number of different pack types and sizes. This has a huge economical advantage and reduces manufacturing to one dosage form to replace the various strengths of capsules and tablets.

The flexible patient tailored dosing of phentermine to patients using the liquid dosage form of the various embodiments for appetite suppression as accurately as 1.5 mg per drop for dosages between 1.5 mg and 50 mg daily or when needed intermittently is advantageous in the treatment of obesity by way of a well-tolerated, safe and effective weight loss therapy that can minimize obesity-associated co morbidities to impede the growing global obesity epidemic.

The various embodiments thus make multiple oral capsules and tablets of different strengths redundant and replaces them by one product able to deliver the required dose of phentermine accurately to a patient that can be given as needed taking in account the dose regime per patient to give appetite suppression at the most effective dose level with minimum side effects.

### DETAILED DESCRIPTION OF THE VARIOUS EMBODIMENTS

The preferred embodiments are now described, by way of non-limiting examples only.

### Example 1

### Phentermine Liquid Dosage Form 1—solution

900 mg of phentermine HCl were dissolved in 30 ml of water. In addition to the solvent, the following additives were also added to the liquid dosage form in the amounts indicated:

TABLE 1

| Component | Amount | |
|---|---|---|
| Methyl paraben | 0.0001 | g |
| Propyl paraben | 0.0002 | g |
| Citric acid | 0.02 | g |
| Propylene glycol | 0.1 | ml |
| Water | 1.0 | ml |

The thus resulting liquid dosage form of phentermine had a concentration of 30 mg/ml of phentermine.

30 ml of the liquid was placed in an amber Type III Ph Eur glass bottle with a polypropylene screw-on cap with a dropper insert that will allow for accurate droplet administration.

The bottled liquid dosage form was retained for administration to a patient to be treated.

### Example 2

### Phentermine Liquid Dosage Form 1—Suspension

450 mg of phentermine HCl were suspended in 20 ml of ethylalcohol. In addition to the liquid, the following additives were also added to the liquid dosage form in the amounts indicated:

TABLE 2

| Component | Amount | |
|---|---|---|
| Methyl paraben | 0.0005 | g |
| Propyl paraben | 0.0001 | g |
| Disodium orthophosphate dihydrate | 0.05 | g |
| Propylene glycol | 0.1 | ml |
| Ethylalcohol | 1.0 | ml |

The thus resulting liquid dosage form of phentermine had a concentration of 22.5 mg/ml of phentermine.

20 ml of the liquid was placed in an amber Type III Ph Eur glass bottle, or a Polyethylene, Polypropylene, HDPE or other suitable container with a polypropylene, or other suitable screw-on cap with a dropper insert that will allow for accurate droplet administration.

The bottled liquid dosage form was retained for administration to a patient to be treated.

### Example 3

### Administration to a Patient

The liquid dosage form of phentermine as prepared in Example 1 above may be administered to an obese patient for the treatment of obesity, appetite suppression or related conditions.

The administration regime requires that 15 to 45 mg of phentermine be administered to the patient twice daily and thus to administer 15 to 45 mg of phentermine only 10 to 30 drops are required of a 30 mg/ml phentermine concentration in the liquid dosage form.

This regime is repeated for a prescribed period, however, the dosage quantity can be adjusted up or down in incremental steps of 1.5 mg of phentermine which is equivalent to 1 drop of the liquid dosage form. This will minimize side-effects and optimize dosage flexibility.

Patient compliance and dosage accuracy is increased over the conventional tablet or other solid dosage form treatment regime.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A pharmaceutical composition liquid dosage form of phentermine, the dosage form comprising a liquid carrier and suspended or dissolved therein one or more pharmaceutically active ingredients selected from the group consisting of phentermine, phentermine salts, phentermine bases, and combinations thereof.

2. The pharmaceutical composition liquid dosage form as claimed in claim 1, wherein the liquid carrier is selected from the group consisting of water, oil, alcohol, and a combination of water and alcohol.

3. The pharmaceutical composition liquid dosage form as claimed in claim 2, wherein the liquid carrier comprises ethanol.

4. The pharmaceutical composition liquid dosage form as claimed in claim 1, wherein the liquid carrier is an oil.

5. The pharmaceutical composition liquid dosage form as claimed in claim 1, wherein the liquid dosage form is selected from the group consisting of elixirs, liquids, solutions, suspension in an aqueous liquid, suspension in a non-aqueous liquid, emulsions, liquid dispersions for oral administration, syrups for oral administration, emulsions for oral administration, solutions for oral administration, and suspensions for oral administration.

6. The pharmaceutical composition liquid dosage form as claimed in claim 1, wherein the one or more pharmaceutically active ingredients comprise one or more of phentermine HCl and other salts of phentermine.

7. The pharmaceutical composition liquid dosage form as claimed in claim 1, wherein the phentermine is covalently bound to a chemical moiety, wherein the chemical moiety provides release of phentermine at a rate where a level of phentermine is within a therapeutic range but below toxic levels over a period of time of 8-24 hours.

8. The pharmaceutical composition liquid dosage form as claimed in claim 1, wherein a concentration of phentermine in the liquid carrier is selected such that an amount of milligrams of phentermine, phentermine salt, and/or phentermine base, which is to be administered, can be determined from a number of drops of the pharmaceutical composition liquid dosage form administered.

9. The pharmaceutical composition liquid dosage form as claimed in claim 8, wherein the concentration of phentermine is from 10 to 100 mg per 1 ml of liquid dosage form.

10. The pharmaceutical composition liquid dosage form as claimed in claim 9, wherein the concentration of phentermine is from 15 mg/ml to 40 mg/ml.

11. The pharmaceutical composition liquid dosage form as claimed in claim 1, further comprising at least one surface-active agent and in a liquid dosage form selected from the group consisting of a draught in water, a draught in a syrup, a non-aqueous suspension, a non-aqueous suspension comprising at least one suspending agent, a suspension in water, and a suspension in a syrup.

12. A phentermine containing liquid composition administration apparatus, comprising a container and a dropper sized and dimensioned to permit a phentermine containing liquid dosage form to be accurately administered in from 1 to 50 mg phentermine per administration thereof.

13. The phentermine containing liquid composition administration apparatus as claimed in claim 12, wherein the dropper is calibrated such that 20 drops of the phentermine containing liquid dosage form are 1 ml, which is equivalent to from 1 to 100 mg phentermine per ml.

14. A method of administering at least one of phentermine, phentermine salts or phentermine bases, to a patient in need thereof, the method comprising administering a preselected number of drops of a pharmaceutical composition liquid dosage form of at least one of phentermine, phentermine salts or phentermine bases to a patient, thereby administering a preselected dose to the patient.

15. A method as claimed in claim 14, comprising administering 10 drops, equivalent to 0.5 ml, of a 30 mg/ml composition in fractional doses more than once daily.

16. A method as claimed in claim 14, comprising administering the pharmaceutical composition liquid dosage form to a patient to be treated for obesity or as an appetite suppressant or related condition in accordance with a prescribed dosage regime.
